(19) United States
(12) Patent Application Publication
(10) Pub. No.: US 2025/0268516 A1
(43) Pub. Date: Aug. 28, 2025

(54) WOUND ASSESSMENT METHOD AND SYSTEM

(71) Applicant: National Cheng Kung University, Tainan (TW)

(72) Inventors: Yuan-Yu Hsueh, Tainan (TW); Shyh-Hau WANG, Tainan (TW); Chia-Ling WU, Tainan (TW); Yu-Lin CHEN, Tainan (TW); Yi-Syuan SHIN, Tainan (TW)

(21) Appl. No.: 19/058,483

(22) Filed: Feb. 20, 2025

Related U.S. Application Data
(60) Provisional application No. 63/558,189, filed on Feb. 27, 2024.

(51) Int. Cl. A61B 5/00; G06T 5/70; G06T 5/80; G06T 7/00; G06T 7/143; G06T 7/90; G16H 30/40; G16H 50/30

(52) U.S. Cl. CPC A61B 5/445; A61B 5/0082; G06T 5/70; G06T 5/80; G06T 7/0012; G06T 7/143; G06T 7/90; G16H 30/40; G16H 50/30; A61B 2560/0228; G06T 2207/10004; G06T 2207/20076; G06T 2207/20081; G06T 2207/30088; G06T 2207/30096

(57) ABSTRACT

A wound assessment method is provided. The wound assessment method includes receiving an image that includes an L-shaped color calibration card and a wound; performing an image preprocessing on the image, based on the L-shaped color calibration card, to obtain an adjusted image, where the image preprocessing includes a distortion correction and a color calibration; obtaining a wound image excluding the L-shaped color calibration card, based on the adjusted image, where the wound image includes a plurality of regions; and classifying each of the plurality of regions of the wound image into one of wound tissue types, to obtain a wound tissue type distribution of the wound, where the wound tissue types include a granulation tissue, a slough tissue, and an eschar tissue.

### CROSS-REFERENCE TO RELATED APPLICATION(S)

The present application claims the benefit of and priority to U.S. Provisional Patent Application Ser. No. 63/558,189, filed on Feb. 27, 2024, entitled “A system for image processing, differentiating, and recording of the chronic wound”, the contents of which are hereby incorporated herein fully by reference into the present application for all purposes.

### FIELD

The present disclosure generally relates to an image assessment method and system, and more particularly, to a wound assessment method and system.

### BACKGROUND

Clinically, when wounds are not properly cared for and treated, they could lead to various serious complications and, in severe cases, pose a risk of mortality. For example, diabetic patients often exhibit symptoms of vascular disease, resulting in wounds that are difficult to heal and prone to repeated infections, with severe cases even facing the risk of amputation. However, when clinically dealing with and treating highly complex wounds, it is necessary to rely on doctors to assess and diagnose the wound first, in order to classify the wounds and propose appropriate treatment strategies, which not only takes more time to diagnose, but may also induce errors in diagnostic results due to the subjective opinions from different doctors, and may delay treatment. Therefore, providing an effective method for wound assessment to facilitate comprehensive care for wounds is a technical challenge with significant clinical application value.

### SUMMARY

In view of the above, the present disclosure provides a wound assessment method and a wound assessment system utilizing the above-mentioned method. By employing the wound assessment method, a subject's wound area and wound tissue types may be effectively evaluated based on the target wound image, thus facilitating the early planning of subsequent medical treatments.

According to a first aspect of the present disclosure, a wound assessment method is provided. The wound assessment method including receiving an image, the image comprising an L-shaped color calibration card and a wound; performing an image preprocessing on the image, based on the L-shaped color calibration card, to obtain an adjusted image, the image preprocessing including a distortion correction and a color calibration; obtaining a wound image excluding the L-shaped color calibration card, based on the adjusted image, the wound image comprising a plurality of regions; and classifying each of the plurality of regions of the wound image into one of wound tissue types to obtain a wound tissue type distribution of the wound, the wound tissue types including a granulation tissue, a slough tissue, and an eschar tissue.

In an implementation of the first aspect of the present disclosure, the wound is located on an open side of a L-shaped structure ruler of the L-shaped color calibration card in the image.

In another implementation of the first aspect of the present disclosure, the distortion correction is performed on the image based on an angle of the L-shaped color calibration card in the image.

In another implementation of the first aspect of the present disclosure, the L-shaped color calibration card includes an L-shaped structure ruler and a plurality of color calibration elements, the plurality of color calibration elements comprises a red pattern, a yellow pattern, a blue pattern, a green pattern, and four progressive grayscale patterns.

In another implementation of the first aspect of the present disclosure, the color calibration is performed on the image based on the red pattern, yellow pattern, blue pattern, and green pattern of the L-shaped color calibration card in the image.

In another implementation of the first aspect of the present disclosure, the wound assessment method further including performing a white balance on the image using the four progressive grayscale patterns.

In another implementation of the first aspect of the present disclosure, the wound assessment method further including converting a color space of the adjusted image to obtain a converted color-space adjusted image; performing a color quantization on the converted color-space adjusted image to reduce a complexity of raw pixels in the converted color-space adjusted image and obtain a color-quantized adjusted image; and performing a denoising processing on the color-quantized adjusted image.

In another implementation of the first aspect of the present disclosure, the wound assessment method further including inputting the adjusted image into an image segmentation model to obtain the wound image excluding the L-shaped color calibration card; inputting the wound image into a tissue segmentation model to obtain a wound tissue image; and determining the wound tissue type distribution of the wound based on the wound tissue image and the wound tissue types.

In another implementation of the first aspect of the present disclosure, the wound assessment method further including performing an area evaluation on the wound tissue image based on the L-shaped color calibration card.

In another implementation of the first aspect of the present disclosure, the wound assessment method further including optimizing the wound tissue image by applying a conditional random field.

In another implementation of the first aspect of the present disclosure, a method for establishing the image segmentation model includes obtaining a plurality of first annotated result images, the plurality of first annotated result images including a plurality of first preprocessed images, each of the plurality of first preprocessed images including a wound category label and a non-wound category label; storing the plurality of first annotated result images as a first dataset; and training the image segmentation model by inputting the first dataset, where the plurality of first preprocessed images is corrected based on the L-shaped color calibration card.

In another implementation of the first aspect of the present disclosure, the image segmentation model includes a Feature Pyramid Network (FPN).

In another implementation of the first aspect of the present disclosure, a method for establishing the tissue segmentation model includes obtaining a plurality of second annotated result images, the plurality of second annotated result images including a plurality of second preprocessed images, each of the plurality of second preprocessed images comprising at least one of wound tissue type labels, where the wound tissue type labels include a granulation tissue label, a slough tissue label, and an eschar tissue label; storing the second annotated result images as a second dataset; and training the tissue segmentation model by inputting the second dataset, where the plurality of second preprocessed images is corrected based on the L-shaped color calibration card.

In another implementation of the first aspect of the present disclosure, the tissue segmentation model includes a Feature Pyramid Network (FPN).

In another implementation of the first aspect of the present disclosure, the wound assessment method further including calculating a wound healing score based on the wound image.

According to a second aspect of the present disclosure, a wound assessment system is provided. The wound assessment system including at least one processor; and at least one memory coupled to the at least one processor and storing at least one computer-executable instruction that, when executed by the at least one processor, cause the wound assessment system to execute the wound assessment method of the first aspect of the present disclosure.

### BRIEF DESCRIPTION OF THE DRAWINGS

This patent or application file contains at least one drawing executed in color. Copies of this patent or patent application publication with color drawing(s) will be provided by the Office upon request and payment of the necessary fee. The present disclosure will be better understood from the following detailed description read in light of the accompanying drawings, where:

FIG. 1 is a schematic diagram illustrating an L-shaped color calibration card according to an example implementation of the present disclosure.

FIG. 2 is a block diagram overviewing a wound assessment system according to an example implementation of the present disclosure.

FIG. 3 is a schematic diagram illustrating the L-shaped color calibration card and a wound according to an example implementation of the present disclosure.

FIG. 4 is a flowchart of a wound assessment method according to an example implementation of the present disclosure.

FIG. 5 is a schematic diagram illustrating a distortion correction of a wound using the L-shaped color calibration card according to an example implementation of the present disclosure.

FIG. 6 is an overview schematic diagram illustrating an image edge optimization according to an example implementation of the present disclosure.

FIG. 7 is a flowchart of a method for establishing an image segmentation model according to an example implementation of the present disclosure.

FIG. 8A is a schematic diagram illustrating image segmentation according to an example implementation of the present disclosure.

FIG. 8B is a schematic diagram illustrating tissue segmentation according to an example implementation of the present disclosure.

FIG. 9 is a flowchart of a method for establishing a tissue segmentation model according to an example implementation of the present disclosure.

FIG. 10 is an overview schematic diagram illustrating area evaluation according to an example implementation of the present disclosure.

### DETAILED DESCRIPTION

The detailed description provided below in connection with the appended drawings is intended as a description of the present examples and is not intended to represent the only forms in which the present examples may be constructed or utilized. The description sets forth the functions of the examples and the sequence of steps for constructing and operating the examples. However, the same or equivalent functions and sequences may be accomplished by different examples.

For convenience, certain terms employed in the specification, examples, and appended claims are collected here. Unless otherwise defined herein, scientific, and technical terminologies employed in the present disclosure shall have the meanings that are commonly understood and used by one of ordinary skill in the art. Also, unless otherwise required by context, it will be understood that singular terms shall include plural forms of the same, and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Terms such as “at least one embodiment”, “one embodiment”, “multiple embodiments”, “different embodiments”, “some embodiments”, “present embodiment”, and the like may indicate that an embodiment of the present disclosure so described may include a particular feature, structure, or characteristic, but not every possible embodiment of the present disclosure must include a particular feature, structure, or characteristic. Furthermore, repeated use of the phrases “in one embodiment”, “in the embodiment”, and so on does not necessarily refer to the same embodiment, although they may be identical. Furthermore, the use of phrases such as “embodiments” in connection with “the present disclosure” does not imply that all embodiments of the present disclosure necessarily include a particular feature, structure, or characteristic, and should be understood as “at least some embodiments of the present disclosure” include the particular feature, structure, or characteristic described.

Additionally, for the purposes of explanation and non-limitation, specific details such as functional entities, techniques, protocols, standards, and the like are set forth for providing an understanding of the described technology. In other examples, detailed disclosure of well-known methods, technologies, systems, architectures, and the like are omitted so as not to obscure the disclosure with unnecessary details.

The terms “first”, “second”, and “third” in the description of the present disclosure and the above-mentioned drawings are used to distinguish different objects, rather than to describe a specific order.

Furthermore, the term “comprising” and any variations thereof are intended to cover non-exclusive inclusions and may refer to “including but not necessarily limited to”, which specifically indicates open-ended inclusion or membership in the so-described combination, group, series, and the equivalent. For example, a process, method, system, product, or device that includes a series of steps or modules is not limited to the listed steps or modules, but optionally also includes steps or modules that are not listed, or optionally also includes other steps or modules that are inherent to those processes, methods, products, or devices.

The implementations of the present disclosure are described below with reference to the accompanying drawings.

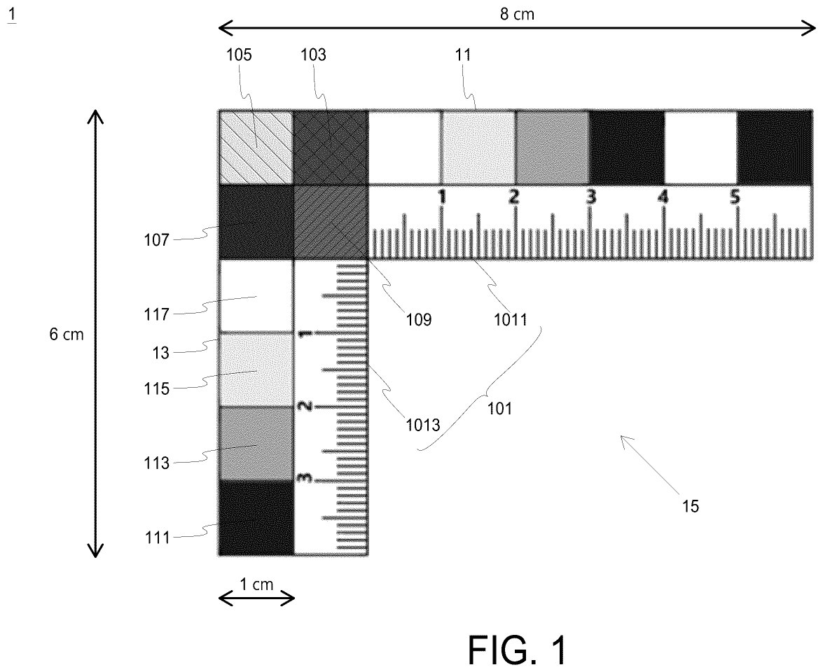

FIG. 1 is a schematic diagram illustrating an L-shaped color calibration card according to an example implementation of the present disclosure.

Referring to FIG. 1. the L-shaped color correction card 1 includes a L-shaped structure ruler 101 and a plurality of color calibration elements (103, 105, 107, 109, 111, 113, 115, 117). The L-shaped color calibration card 1 includes a long side 11 and a short side 13. In some implementations, the angle between the long side 11 and the short side 13 is 90 degrees.

In some implementations, the L-shaped structure ruler 101 includes a horizontal ruler 1011 and a vertical ruler 1013, where the length of the horizontal ruler 1011 may be 6 cm, which not limited thereto, and the length of the vertical ruler 1013 may be 4 cm, which is not limited thereto. In some implementations, the angle formed between the horizontal ruler 1011 and the vertical ruler 1013 is 90 degrees. It should be noted that the determination of the horizontal ruler and the vertical ruler depend on the orientation of the L-shaped color calibration card 1 during image capturing.

In some implementations, the L-shaped structure ruler 101 of the L-shaped color calibration card 1 may form an open side 15.

In some implementations, the long side 11 of the L-shaped color calibration card 1 may be 8 cm, which is not limited thereto, and the short side 13 of the L-shaped color correction card 1 may be 6 cm, which is not limited thereto. The color calibration elements (103, 105, 107, 109, 111, 113, 115, 117) are all square-shaped, and the side length of each color calibration element may be 1 cm, which is not limited thereto.

In some implementations, the color calibration elements (103, 105, 107, 109, 111, 113, 115, 117) include a red pattern 103, a yellow pattern 105, a blue pattern 107, a green pattern 109, and four progressive grayscale color patterns (111, 113, 115, 117), where the four progressive grayscale color patterns (111, 113, 115, 117) include a black pattern 111 and a white pattern 117, as well as a gray pattern 113 and an gray-white pattern 115. The gray pattern 113 and the gray-white pattern 115 are between the colors of the black pattern 111 and the white pattern 117. In some implementations, the L-shaped structure ruler 101 is adjacent to one of the color calibration elements (103, 105, 107, 109). In some implementations, the L-shaped structure ruler 101 is adjacent to the green pattern 109.

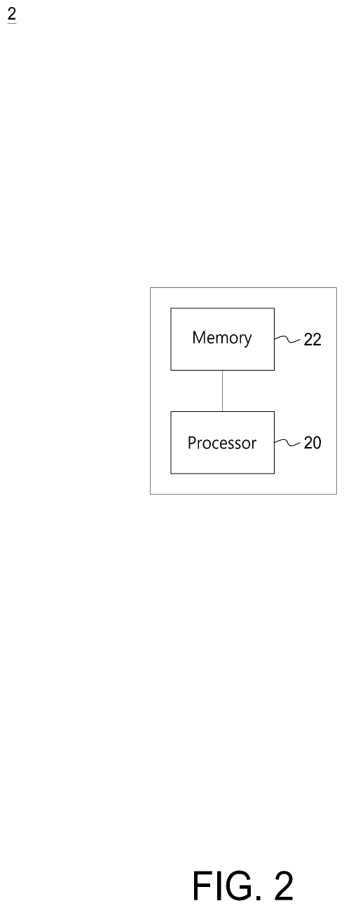

FIG. 2 is a block diagram overviewing a wound assessment system according to an example implementation of the present disclosure.

In some implementations, the wound assessment system 2 includes a processor 20 and a memory 22.

In some implementations, the processor 20 is responsible for running the main computation process and related control logic for algorithms, such as deep learning. The processor 20 may be implemented through a central processing unit (CPU), or may be implemented using other programmable general-purpose or special-purpose microprocessors, digital signal processors (DSPs), programmable controllers, application-specific integrated circuits (ASICs), programmable logic devices (PLDs), or other similar components or combinations of these components.

In some implementations, the wound assessment system 2 may include a memory 22 that stores computer-executable instructions. The memory 22 may include any form of fixed or removable random access memory (RAM), read-only memory (ROM), flash memory, hard disk, or other similar components, or combinations of these components. The memory is used to store executable instructions that enable the processor 20 to execute the instructions, allowing the wound assessment system 2 to perform the various operations described in this disclosure.

In some implementations, the wound assessment system 2 may further include a display device (not shown in the figure) that may be implemented using a liquid crystal display (LCD), light-emitting diode (LED) display, field emission display (FED), or other types of displays, to output various information to the user. In some implementations, the display device may include a speaker, communication interface, or a combination of these components. In other words, this disclosure does not limit the form of the output, and one skilled in the art may design the display device to output various types of information according to their needs.

In some implementations, the wound assessment system 2 may also include an image capture device (not shown in the figure), which is used to capture the target image. The image capture device may be any photographic device used for capturing the target image.

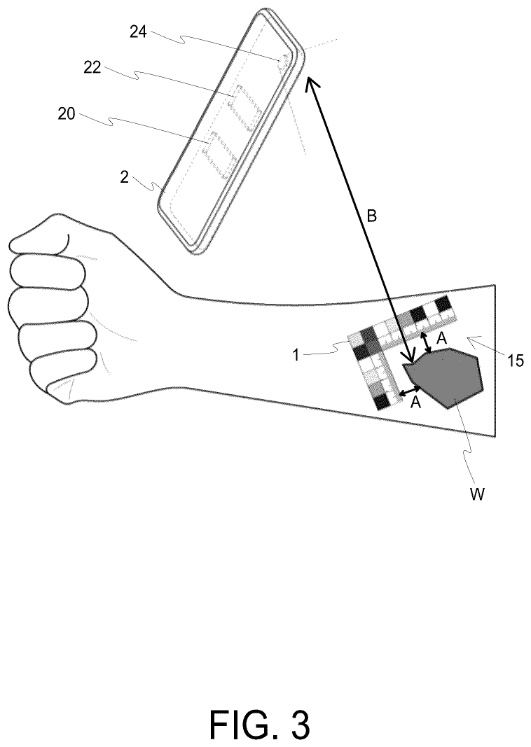

FIG. 3 is a schematic diagram illustrating the L-shaped color calibration card and a wound according to an example implementation of the present disclosure.

Please refer to FIG. 3. In some implementations, when capturing the image, the L-shaped color calibration card 1 must be placed at an edge of the wound W. Specifically, the wound W needs to be positioned at the open side 15 of the L-shaped structure ruler 101 of the L-shaped color calibration card 1. Meanwhile, there is a distance A between the L-shaped color calibration card 1 and the edge of the wound W, where the distance A is between 1 cm to 2 cm.

In some implementations, the wound assessment system 2 includes a processor 20, a memory 22, and an image capturing device 24. There is a distance B between the image capturing device 24 and the wound W, where the distance B is between 5 cm to 10 cm.

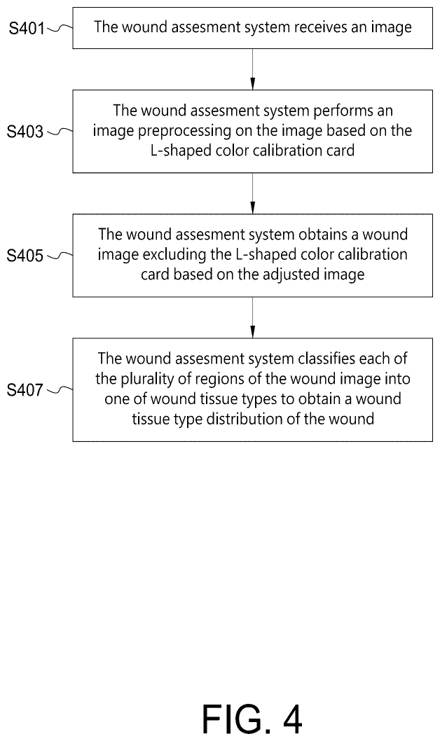

FIG. 4 is a flowchart of a wound assessment method according to an example implementation of the present disclosure.

Referring to FIG. 4, in step S401, the wound assessment system receives an image. The image may also be provided by the wound assessment system. In some implementations, the wound assessment system captures an image of the subject's wound to provide or receive a target image. The target image is analyzed by the processor 20. The target image includes the L-shaped color calibration card and the wound.

In some implementations, the image may be provided by a wound image database.

Please referring to FIG. 4, in step S403, the wound assessment system performs an image preprocessing on the image, based on the L-shaped color calibration card.

Since images are typically not captured in a fixed manner, a distortion correction is required on the image to ensure that the wound size in the image closely approximates the actual wound size. Additionally, capturing images under different lighting conditions may affect the color accuracy of the image. Therefore, a color calibration is also necessary to eliminate color deviations in the image. For clarity, the details of steps S405 and S407 are not described herein will be provided in subsequent sections.

In some implementations, to address the above problems, an image preprocessing may be performed. Specifically, the processor in the wound assessment system may perform the image preprocessing on the image, based on the L-shaped color calibration card in the image, to obtain an adjusted image. The method for image preprocessing performed by the processor includes applying the distortion correction to the image, based on the angle of the L-shaped color calibration card, and performing the color calibration on the image using the red pattern, yellow pattern, blue pattern, and green pattern, thus obtaining the adjusted image.

In some implementations, the method for image preprocessing performed by the processor includes applying distortion correction to the image, based on the angle of the L-shaped structure ruler, and performing the color calibration on the image using the red pattern, yellow pattern, blue pattern, and green pattern, thus obtaining the adjusted image. In some implementations, the method for applying distortion correction to the image, using the L-shaped structure ruler, may include utilizing a rotation matrix equation or a perspective transformation matrix equation.

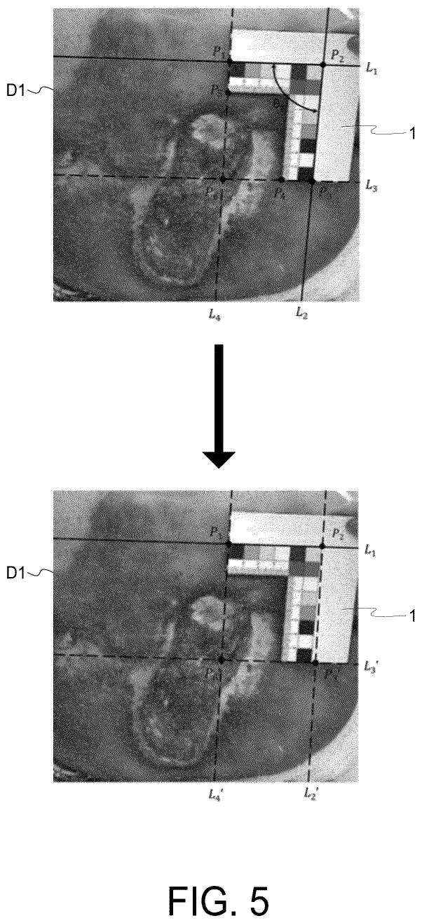

FIG. 5 is a schematic diagram illustrating a distortion correction of a wound using the L-shaped color calibration card according to an example implementation of the present disclosure.

Referring to FIG. 5, in some implementations, the method performed by the processor for applying distortion correction to the image involves detecting reference points P1, P2, P3, P4, and P5 on the L-shaped color calibration card 1 in image D1. First, line L1 passing through reference points P1 and P2, line L2 passing through reference points P2 and P3 are detected. Then, the angle θ₁ between lines L1 and L2 is calculated to determine whether it equals 90 degrees. If the angle θ₁ is not 90 degrees, detect the line L3 passing through reference points P3 and P4, line L4 passing through reference points P1 and P5 on the L-shaped color calibration card 1 in the image, and identify the intersection points P1, P2, P3, and P6 among lines L1, L2, L3, and L4. Subsequently, in image D1, the following are detected: line L3′, parallel to line L1 and passing through reference point P3; line L2′, perpendicular to line L1 and passing through reference point P2; and line L4′, perpendicular to line L1 and passing through reference point P1. Identify the intersection points P3′ and P6′ formed by each pair of lines among lines L2′, L3′, and L4′. Using these six points (P1, P2, P3, P6, P3′, P6′), a perspective transformation matrix M is calculated. Finally, the perspective transformation matrix is applied to the image to perform the perspective transformation, thus, achieving the distortion correction. This ensures the target region in the image (e.g., the wound) may be closer to the real size of the target region. The perspective transformation formula is represented in equation (1).

In homogeneous coordinates, a 2D point (x, y) may be extended into a three-dimensional vector

$$[ \begin{matrix}x \\ y \\ 1\end{matrix} ] .$$

The perspective transformation matrix M may map the point (x, y) to a new point (x′, y′).

$$\begin{matrix}M = [ \begin{matrix}{h}_{11} & {h}_{12} & {h}_{13} \\ {h}_{21} & {h}_{22} & {h}_{23} \\ {h}_{31} & {h}_{32} & {h}_{33}\end{matrix} ] \\ [ \begin{matrix}{x}^{′} \\ {y}^{′} \\ {w}^{′}\end{matrix} ] = [ \begin{matrix}{h}_{11} & {h}_{12} & {h}_{13} \\ {h}_{21} & {h}_{22} & {h}_{23} \\ {h}_{31} & {h}_{32} & {h}_{33}\end{matrix} ] [ \begin{matrix}x \\ y \\ 1\end{matrix} ]\end{matrix} \qquad Equation ( 1 )$$

In some implementations, the processor may detect the angle between the horizontal ruler and the vertical ruler of the L-shaped structure ruler 101 in the image. It should be noted that the horizontal and vertical rulers described here are determined based on the placement of the L-shaped structure ruler during imaging. For example, the ruler parallel to the x-axis of the image is referred to as the horizontal ruler, and the ruler parallel to the y-axis of the image is referred to as the vertical ruler. Then, the angle between the horizontal and vertical rulers is calculated to check whether the angle is 90 degrees. If the angle is not 90 degrees, indicating that image distortion occurred due to image capturing errors, and the image is then corrected using the perspective transformation equation.

As described above, designing the L-shaped color calibration card as an L-shaped structure, or as a two-dimensional color calibration card, is essential for performing image distortion correction. Specifically, two-dimensional rulers have significant advantages over one-dimensional rulers, primarily in terms of calibration capability and application range. First, a two-dimensional ruler provides reference points in two directions, enabling more comprehensive image calibration, whereas a one-dimensional ruler may only provide reference in a single direction and cannot achieve complete two-dimensional calibration. This allows a two-dimensional ruler, such as the L-shaped structure ruler, to offer more spatial reference points, effectively correcting perspective distortion and geometric distortion. Additionally, the calibration effect of the L-shaped structure ruler is more uniform and extensive. Compared to a one-dimensional ruler, which mainly corrects the area near the ruler, a two-dimensional ruler may perform more balanced calibration across the entire image, even effectively correcting parts of the image far from the ruler. This advantage is particularly noticeable when handling large or complex images. Finally, from a practical perspective, the L-shaped structure ruler allows for measurements in two directions, greatly increasing its utility in various application scenarios. In contrast, a one-dimensional ruler may only measure in a single direction, limiting its scope of use. This multi-directional measurement capability makes the L-shaped structure ruler a more comprehensive tool that could meet a wider range of measurement and calibration needs.

In some implementations, the accuracy of the target area measurement is compared between images with and without distortion correction. For example, the actual target area is 8.48 cm². Under conditions of oblique imaging, the processor directly calculates the target area in the image as 5.12 cm². However, when the processor first performs the distortion correction on the image and then calculates the target area, the result is 9.19 cm². This demonstrates that by first performing the distortion correction on the image and then calculating the target area, the resulting measurement is closer to the actual value.

In some implementations, the processor performs color calibration on an image by detecting the red pattern 103, yellow pattern 105, blue pattern 107, green pattern 109, black pattern 111, and white pattern 117 on the L-shaped color calibration card 1. Specifically, the red pattern 103, yellow pattern 105, blue pattern 107, green pattern 109, black pattern 111, and white pattern 117 correspond to their reference RGB values, as shown in Table 1. Table 1 provides the reference RGB values for the color calibration card. The processor uses the known reference values of these patterns, such as red pattern 103, yellow pattern 105, blue pattern 107, green pattern 109, black pattern 111, gray pattern 113, gray-white pattern 115, and white pattern 117, along with a Color Correction Matrix (CCM), to correct the colors in the image. This ensures that the measured color values may be transformed into the corresponding known reference values.

In some implementations, the equation for the color correction matrix is shown in Equation (2), where C is the 3×3 color correction matrix, R is the reference color matrix, and E is the actual color matrix from the image. Given the reference color matrix and the actual color matrix from the image, the color correction matrix C could be calculated.

$$\begin{matrix}C = [ \begin{matrix}{C}_{R 1} & {C}_{G 1} & {C}_{B 1} \\ {C}_{R 2} & {C}_{G 2} & {C}_{B 2} \\ {C}_{R 3} & {C}_{G 3} & {C}_{B 3}\end{matrix} ] ; R = [ \begin{matrix}{R}_{1}^{′} & {G}_{1}^{′} & {B}_{1}^{′} \\ ⋮ & ⋮ & ⋮ \\ {R}_{n}^{′} & {G}_{n}^{′} & {B}_{n}^{′}\end{matrix} ] ; E = [ \begin{matrix}{R}_{1} & {G}_{1} & {B}_{1} \\ ⋮ & ⋮ & ⋮ \\ {R}_{n} & {G}_{n} & {B}_{n}\end{matrix} ] \\ R = E \times C , n \geq 3\end{matrix} \qquad Equation ( 2 )$$

TABLE 1

| Color | Reference RGB values |
|---|---|
| Blue | (0, 0, 255) |
| Red | (255, 0, 0) |
| Green | (0, 255, 0) |
| Yellow | (255, 255, 0) |
| White | (255, 255, 255) |
| Gray-whit | (213, 213, 213 |
| Gray | (129, 129, 129) |
| Black | (0, 0, 0) |

In some implementations, the processor may further include a white balance correction as part of the image preprocessing method. Specifically, the processor may perform brightness correction or white balance correction on the image, based on four progressive grayscale color patterns (111, 113, 115, 117), to eliminate unnatural color casts in the image.

In some implementations, the processor may further convert the color space of the adjusted image to obtain a converted color-space adjusted image. The purpose is to use the CIELab color space, which closely resembles human vision, for subsequent tissue segmentation steps. In the CIELab color space, “L” represents perceived lightness, “a” represents the range of colors from red to green, and “b” represents the range of colors from blue to yellow. Therefore, the processor may convert the RGB color space of the adjusted image into CIEXYZ through linear conversion to obtain the CIEXYZ space image, and then calculate the CIEXYZ space image into CIELab space. The color space conversion equations are shown in Equation (3). The range of L is from 0 to 100, where 0 represents black and 100 represents white. The range of a is from −128 to +127, where positive values represent red and negative values represent green. The range of b is from −128 to +127, where positive values represent yellow and negative values represent blue. Furthermore, the D65 light source may be represented as the most commonly used artificial daylight among standard illuminants.

$$\begin{matrix}\begin{matrix}[ \begin{matrix}X \\ Y \\ Z\end{matrix} ] = [ \begin{matrix}0.607 & 0.174 & 0.200 \\ 0.299 & 0.587 & 0.114 \\ 0. & 0.066 & 1.116\end{matrix} ] \times [ \begin{matrix}R \\ G \\ B\end{matrix} ] \\ L = 116 \times \sqrt[3]{\frac{Y}{{Y}_{0}}} - 16 \\ A = 500 \times [ \sqrt[3]{\frac{X}{{X}_{0}}} - \sqrt[3]{\frac{Y}{{Y}_{0}}} ]\end{matrix} \\ B = 20 \; 0 \times [ \sqrt[3]{\frac{Y}{{Y}_{0}}} - \sqrt[3]{\frac{Z}{{Z}_{0}}} ]\end{matrix} \qquad Equation ( 3 )$$

In some implementations, the captured images may contain noise, such as Gaussian noise or impulse noise, which may affect the results of image segmentation (e.g., wound segmentation) in the images. Therefore, prior to performing the wound segmentation, it is necessary to preprocess the original images to remove noise. In some implementations, the processor may perform distortion correction, color calibration, color space conversion, and color quantization on the original image to obtain a processed image. Subsequently, the processed image is denoised to obtain an adjusted image. For example, the processor may use a bilateral filter for image denoising. The bilateral filter is a nonlinear noise filter that considers both the Euclidean distance and similarity between pixels. This effectively reduces computational complexity while preserving the details and edges of the image, and effectively removes noise. Denoising the image may enhance the accuracy and efficiency of subsequent wound segmentation steps on the image. The equation for the bilateral filter is shown in Equation (4). The h_{b} represents the output image value, m and n are the filter dimensions, c(x_{p,q}, x_{i,j}) represents the geometric distance between two points x_{p,q} and x_{i,j} in the correlation function, and s(ƒ(x_{p,q}), ƒ(x_{i,j})) represents the difference in chromaticity between two points ƒ(x_{p,q}) and θ(x_{i,j}), where x_{p,q} refers to the coordinates of the center point of the filter mask, and x_{i,j} refers to the coordinates of neighboring points around the center point. ƒ(x_{p,q}) is the pixel value at the center point of the filter mask, and ƒ(x_{i,j}) is the pixel value of neighboring points around the center point.

$$\begin{matrix}{h}_{b} ( {x}_{i , j} ) = {k}_{b}^{- 1} ( x ) \times {\sum }_{p = i - m}^{i + m} {\sum }_{q = j - n}^{j + n} f ( {x}_{i , j} ) \times c ( {x}_{p , q} , {x}_{i , j} ) \times s ( f ( {x}_{p , q} ) , f ( {x}_{i , j} ) ) \\ {k}_{b} ( x ) = {\sum }_{p = i - m}^{i + m} {\sum }_{q = j - n}^{j + n} s ( f ( {x}_{p , q} ) , f ( {x}_{i , j} ) ) \times c ( {x}_{p , q} , {x}_{i , j} )\end{matrix} \qquad Equation ( 4 )$$

Referring to FIG. 4, in step S405, the wound assessment system obtains a wound image excluding the L-shaped color calibration card based on the adjusted image.

In some implementations, the processor may input the adjusted image into an image segmentation model to obtain a wound image excluding the L-shaped color calibration card. Specifically, the processor may input the adjusted image into the image segmentation model to segment the adjusted image and extract only the wound area, thus generating the wound image.

In some implementations, the wound image includes a plurality of regions, where each region represents a wound tissue.

Referring to FIG. 4, in step S407, the wound assessment system classifies each of the plurality of regions of the wound image into one of wound tissue types to obtain a wound tissue type distribution of the wound.

In some implementations, the processor may further input the wound image into a tissue segmentation model, the tissue segmentation model classifies each pixel within the wound image into one of the wound tissue types to obtain a wound tissue segmentation result. Subsequently, based on the wound tissue segmentation result and the multiple wound tissue types, a wound tissue type distribution of the wound is determined. In this case, the wound tissue types include a granulation tissue, a slough tissue, and an eschar tissue.

In some implementations, the processor may further input the wound image into a tissue segmentation model, the tissue segmentation mode classifies each pixel in the wound image into one of the wound tissue types to obtain a wound tissue image. Specifically, the wound tissue image may provide information about the wound tissue type of the wound and the wound tissue type distribution of the wound. For example, in some implementations, the wound tissue image may reveal that the wound tissue types of the wound includes granulation tissues and slough tissues, indicating that the wound tissue of this wound includes granulation tissues and slough tissues. Furthermore, the distribution of granulation tissues and slough tissues within the wound may also be determined from the wound tissue image.

However, during tissue segmentation, false positives or the influence of data noise may result in suboptimal tissue segmentation outcomes. Therefore, the processor may perform an image post-processing on the image including wound tissue to enhance the accuracy and practicality of the tissue segmentation outcomes. For example, the processor may optimize images containing wound tissues through Conditional Random Fields (CRFs) to improve the precision of the segmentation edges of each wound tissue in the image.

The processor may optimize the segmentation edges of each region in the image through superpixels. Superpixels is a technique in image processing that aggregates similar pixels, having color similarity and spatial proximity, in an image into a region. This method significantly reduces the dimensionality of the image and eliminates anomalous pixels, thus improving the efficiency and accuracy of image processing. For example, the processor may optimize the edges of each similar region in the image using the Simple Linear Iterative Clustering (SLIC) algorithm within the superpixel approach. The SLIC method is advantageous for its simplicity and efficiency, making it suitable for large-scale image segmentation and processing, and generating superpixels with compact shapes and delivers high-quality segmentation results.

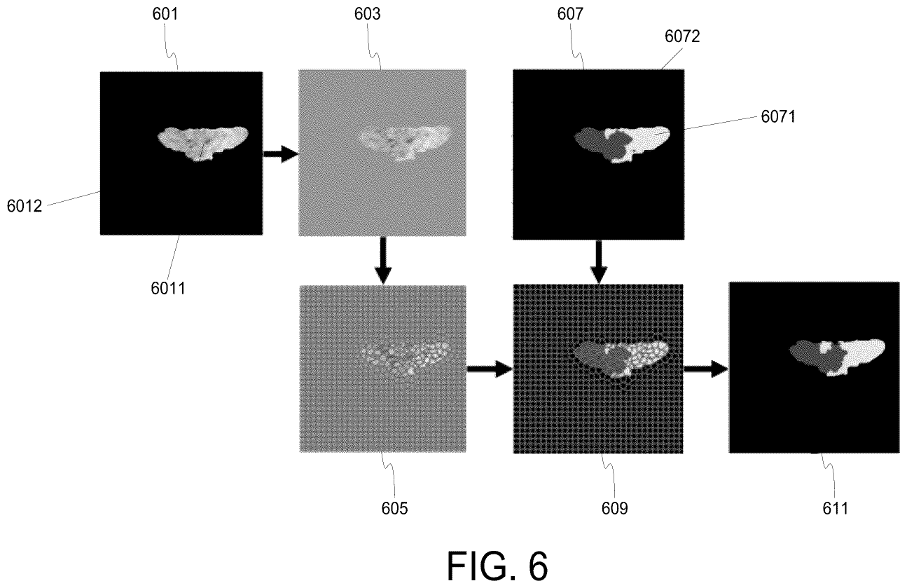

FIG. 6 is an overview schematic diagram illustrating an image edge optimization according to an example implementation of the present disclosure.

Referring to FIG. 6, in some implementations, the process of optimizing image edges using superpixels may include six steps. First, the image segmentation model is used to segment the wound area and non-wound area in the images, and then a wound image 601 is obtained for further processing. The wound image 601 may be a distinguished wound area image. The wound image 601 includes a wound area 6011 and a background 6012. Next, the color of the background 6012 of the wound image 601 is changed from black to green to obtain an adjusted background color wound image 603. The background color is changed from black to green because the wound itself may contain black areas, which could affect the accuracy of pixel region determination when using SLIC. Subsequently, the RGB color space of the adjusted background color wound image 603 is converted to the CIELab color space, and a SLIC processing is applied. During this step, the SLIC algorithm is applied with different values of K, ranging from 1000 to 2000, to compare overall optimization accuracy, thus resulting in a SLIC image 605. In some implementations, setting the value of K to 1000 improves the optimization accuracy of the image while reducing computation time. Simultaneously, the wound image 601 is input into the tissue segmentation model to obtain a wound tissue image 607. From the wound tissue image 607, the wound tissue types of the wound area 6011 and the distribution of the wound tissue types of the wound area 6011 could be determined. For example, the wound tissue types in the wound area 6011 may include slough tissues 6071 and granulation tissues 6072, with the slough tissues 6071 primarily located to the right of the granulation tissues 6072.

Next, the wound tissue image 607 is overlaid with the SLIC image 605 to obtain the overlapping image 609. Finally, edge optimization is performed on the overlapping image 609. In the edge optimization method, pixel selection is based on the proportion of wound tissues in each area. Specifically, if the proportion of the three wound tissue types exceeds 75% of the entire region, the wound tissue type with the highest proportion will fill the entire region, which means that the entire region is adjusted to match the pixels of the wound tissue type with the highest proportion. Conversely, if the proportion of the three wound tissue types is less than 75%, indicating that the background constitutes a larger proportion, the entire region is assigned the pixels of the background. After comparing all K regions, an edge-optimized predicted image 611 is obtained.

In some implementations, the processor may utilize morphological methods to fill holes in the image. Specifically, the processor may perform basic morphological operations, such as dilation, erosion, opening, and closing, to remove noise, fill holes, and smooth edges in the wound tissue image.

Additionally, in some implementations, the processor may perform an area evaluation of the wound tissue image based on the L-shaped color calibration card. The area evaluation calculates the area of each wound tissue type within the wound tissue image to derive wound information. Specifically, the wound information may include the size of the wound area, the types of wound tissue (e.g., granulation tissue, slough tissue, or eschar tissue), the distribution of wound tissue types, and the area size of each wound tissue types.

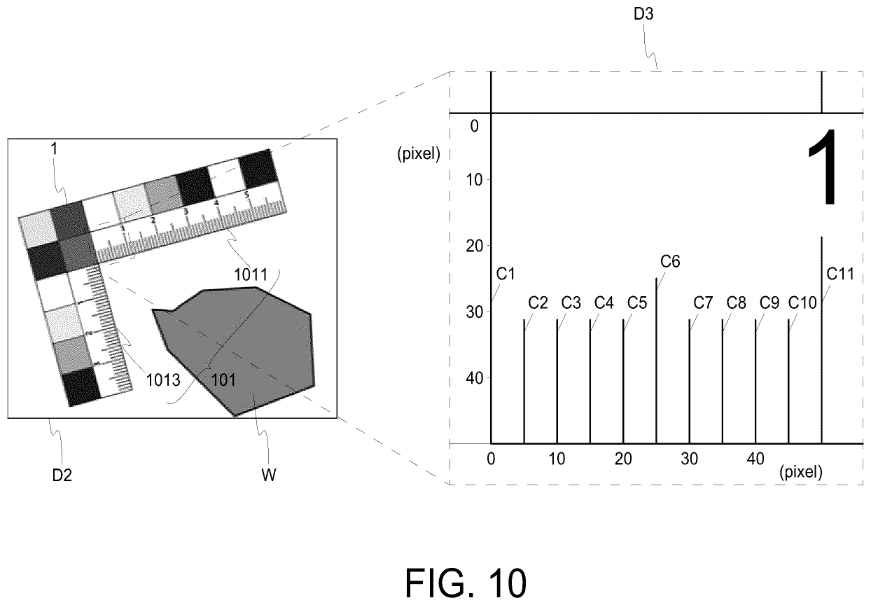

FIG. 10 is an overview schematic diagram illustrating area evaluation according to an example implementation of the present disclosure.

Referring to FIG. 10, in some implementations, the processor may perform area evaluation on the wound image or the wound tissue image. The area evaluation method may involve first detecting the color calibration element adjacent to the L-shaped structure ruler 101 on the L-shaped color calibration card 1 in image D2. A portion of the L-shaped structure ruler 101 near the color calibration element is then cropped as a rectangular image. In some implementations, the color calibration element adjacent to the L-shaped structure ruler 101 may be a green pattern. A portion of the L-shaped structure ruler 101 next to the green pattern is cropped as the rectangular image D3. The rectangular image D3 is then used for calculating the ratio between pixels and actual length. The rectangular image D3 is first subjected to a Laplacian operator for line detection. Subsequently, a Hough transform is applied to identify the most likely lines (C1, C2, C3, C4, C5, C6, C7, C8, C9, C10, C11) within the rectangular image D3. These lines (C1 to C11) represent the scale markings on the L-shaped structure ruler 101. As shown in FIG. 10, in the rectangular image D3, the distance between lines C1 and C11 on the L-shaped structure ruler 101 is approximately 50 pixels, with a total of 10 intervals between these lines. Therefore, the distance between each pair of consecutive lines (e.g., C2 and C3) may be calculated as approximately 5 pixels. Furthermore, in the L-shaped structure ruler 101, if the actual distance between lines C1 and C11 is 1 cm, the actual distance between lines C2 and C3 is 1 mm. Using the distance between each pair of lines, the ratio of pixel values to actual length in rectangular image D3 could be determined. For example, based on the above results, 1 pixel in the rectangular image D3 corresponds to an actual distance of 0.2 mm. By converting the pixel values to actual length using this ratio, the actual area of the wound W in image D2 could be calculated. When the total number of wound pixels is known, the actual wound area may be computed using Equation (5), where a represents the pixel value of the cropped image, and β represents the actual length in centimeters (cm).

$$Area = \frac{Object pixel \times {β}^{2}}{{α}^{2}} \qquad Equation ( 5 )$$

In some implementations, the processor may calculate a wound healing score based on the wound information. The wound healing score is applicable for assessing the numerical progression of wounds over time and incorporates factors, such as wound area, internal wound tissue types, and treatment duration, to provide a score for the wound. Using actual numerical values could offer a more objective assessment compared to relying solely on a physician's past experience to judge wound progression. The wound healing score in the present disclosure adopts the method described in a 2016 publication, by Wang et al., titled “An automatic assessment system of diabetic foot ulcers based on wound area determination, color segmentation, and healing score evaluation”. Using the previously calculated total wound area, the area of segmented wound tissues could be determined, and a weighted area of the wound tissue (WAₙ) is computed using a weighted approach. The calculation is expressed by Equation (6), where A_{G} represents the area of granulation tissue, As represents the area of slough tissue, and A_{N} represents the area of eschar tissue. W_{G} represents the weight of granulation tissue, W_{S} represents the weight of slough tissue, and W_{N} represents the weight of eschar tissue. In the present disclosure, the weights were set to [1, 1.5, 2.5], as the eschar tissue is considered the worst type of tissue, while the granulation tissue represents the beginning stages of healing. Accordingly, the eschar tissue was assigned the highest weight, and the granulation tissue has the lowest weight.

$$W {A}_{n} = {W}_{G} \times {A}_{G} + {W}_{S} \times {A}_{S} + {W}_{N} \times {A}_{N} \qquad Equation ( 6 )$$

Based on the wound score obtained during a current clinical visit, a healing score may be calculated by comparing it with the score from the initial visit. The calculation is represented by Equation (7), where the parameter G is a hyperparameter ranging from 0 to 1, used to set the judgment of the healing score. In some implementations, the method follows the approach described in a 2015 publication, by L. Wang et al., titled “An Automatic Assessment System of Diabetic Foot Ulcers Based on Wound Area Determination, Color Segmentation, and Healing Score Evaluation,” where G could be set to 0.4. The healing score (Sₙ) ranges from 0 to 10, with 0 indicating poor healing progression and 10 representing nearly complete healing. Additionally, all patients begin with a healing score of 5 at their initial clinical visit. WA₀ is the weighted area of the wound at the first clinical visit, and WAₙ is the weighted area of the wound at the nth clinical visit.

$${S}_{n} = 1 - \frac{W {A}_{n} - W {A}_{0}}{W {A}_{0}} \times G \qquad Equation ( 7 )$$

Since the analysis of wounds across different clinical visits is necessary to determine the healing score, only patients with at least three follow-up visits are analyzed. Using the method proposed in the present disclosure, the total wound area and the respective areas of the three wound tissue types could be obtained, measured in square centimeters. These values could then be used to calculate the wound healing score.

In some implementations, the follow-up information for a patient is shown in Table 2, comprising a total of four clinical visits. During the first visit, the wound exhibited eschar tissue. However, by the second follow-up, the eschar tissue had disappeared, and the overall wound area had decreased, resulting in a nearly 70% improvement in the healing score. During the third visit, the overall wound area further decreased, and granulation tissue increased, bringing the healing score to 9. By the fourth follow-up, the wound consisted solely of granulation tissue, with the healing score nearing a state of complete healing.

TABLE 2

| | 1^{st} visit | 2^{nd} visit | 3^{rd} visit | 4^{th} visit |
|---|---|---|---|---|
| Wound Area (cm²) | 4.04 | 2.28 | 1.87 | 0.87 |
| | (100%) | (100%) | (100%) | (100%) |
| Eschar (cm²) | 0.28 | 0 | 0 | 0 |
| | (7%) | (0%) | (0%) | (0%) |
| Slough (cm²) | 3.76 | 2.28 | 0.31 | 0 |
| | (93%) | (100%) | (17%) | (0%) |
| Granulation (cm²) | 0 | 0 | 1.55 | 0.87 |
| | (0%) | (0%) | (83%) | (100%) |
| Healing score | 5 | 8.46 | 9.08 | 9.60 |
| | (ref) | | | |
| Time consuming | N | N + 21 | N + 28 | N + 35 |

Specifically, the primary factor influencing the wound healing score is the change in the overall wound area, followed by the appearance or disappearance of the three wound tissue types. The presence of granulation tissue indicates that the wound is beginning to show signs of healing. Additionally, if the wound healing score does not significantly increase within four weeks, a follow-up consultation is required to reassess the treatment. Conversely, if the current healing score is higher than the previous score, it indicates healing progress, allowing the patient to continue wound care at home without visiting the hospital. Finally, the time required for processing each image using the wound assessment method of the present disclosure is approximately 1.42±0.15 seconds.

Accordingly, the wound assessment system and method of the present disclosure effectively analyze the target wound image data of a subject to evaluate the wound area and wound tissue types based on the information carried by the target wound images. This enables early planning of subsequent medical treatments, thus reducing the risk of complications caused by delayed or improper wound management.

The following section will present the method for establishing the image segmentation model and the tissue segmentation model of the present disclosure.

In some implementations, wound images from hospital patients and open datasets are used, with the datasets divided into training set, validation set, and test set in a ratio of 8:1:1 to train and test the image segmentation model and tissue segmentation model. The wound images from hospital patients include 808 images provided by Dr. Yuan-Yu Hsueh from the Department of Plastic Surgery at the National Cheng Kung University Hospital. These images were collected between January 2021 and February 2023, with 453 images used for training the image segmentation model and tissue segmentation model. These images include wound conditions from the same patient across different follow-up visits. The open dataset is the MICCAI 2021 Foot Ulcer Segmentation Challenge dataset, which contains 800 images, primarily consisting of chronic foot wounds in diabetic patients.

### Image Segmentation Model

In some implementations, the processor could calibrate a plurality of images in the dataset containing the L-shaped color calibration card to obtain a plurality of first preprocessed images.

In some implementations, the processor could perform image preprocessing on images in the dataset based on the L-shaped color calibration card to obtain the first preprocessed images. Specifically, the method for performing the first preprocessing on the images by the processor includes performing distortion correction using the L-shaped color calibration card, converting the color space of the images, and denoising the images. The details of distortion correction, color space conversion, and denoising using the L-shaped color calibration card are as described in the previous sections and will not be repeated herein.

In some implementations, the processor could perform data augmentation on the plurality of first preprocessed images. Specifically, the processor could increase the diversity of training samples by: rotating the plurality of first preprocessed images by 90° using the Rotate90 method; adjusting the brightness and contrast of the images using the BrightnessContrast method; horizontally reflecting the images using the HorizontalFlip method; adding blur effect to the images using the GaussianBlur method; and transposing the images using the Transpose method. In some implementations, the training samples for training the image segmentation model are augmented from 1,287 images to 7,722 images.

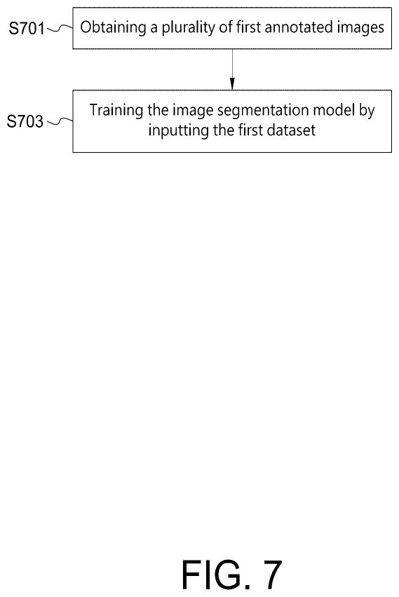

FIG. 7 is a flowchart of a method for establishing an image segmentation model according to an example implementation of the present disclosure.

Please refer to FIG. 7, in step S701, obtaining a plurality of first annotated images.

In some implementations, professional plastic surgeons may assist in annotating the true wound areas in the plurality of first preprocessed images to obtain the plurality of first annotated result images. The wound and non-wound areas in the images could be segmented using superpixels, and the wound and non-wound areas could be labeled using Labelme or other similar open-source software. The corresponding first labels are then assigned to obtain the plurality of first annotated result images. The first labels include wound category label or non-wound category label.

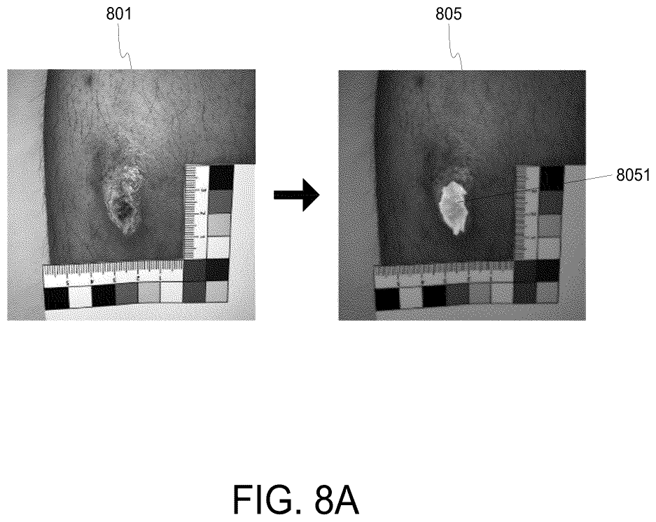

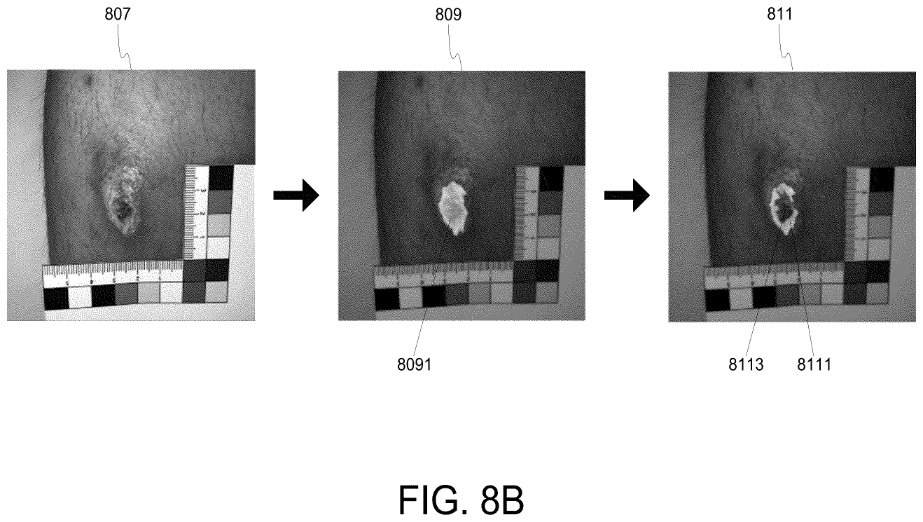

FIG. 8A is a schematic diagram illustrating image segmentation according to an example implementation of the present disclosure.

Please refer to FIG. 8A, the wound and non-wound areas in the first preprocessed image 801 are segmented using superpixels, and corresponding labels (e.g., wound category label or non-wound category label) are assigned to the wound and non-wound areas in the first preprocessed image 801 to obtain the first annotated result image 805. In the first annotated result image 805, region 8051 represents the wound area in the first preprocessed image 801, and the region 8051 is assigned the corresponding wound category label. Meanwhile, the image area in the first annotated result image 805 that do not belong to region 8051 represent the non-wound areas in the first preprocessed image 801, and the image area that do not belong to region 8051 is assigned the corresponding non-wound category label.

Please refer to FIG. 7, in step S703, training the image segmentation model by inputting the first dataset.

In some implementations, the processor may store the first annotated result images as a first dataset and further input the first dataset into an image segmentation model to train the image segmentation model using the first dataset. The purpose of the image segmentation model is to distinguish the wound area and non-wound area within the image.

In some implementations, the image segmentation model may include Fully Convolutional Networks (FCN), the DeeplabV3+ model, the LinkNet model, or the Feature Pyramid Networks (FPN) model. In some implementations, the image segmentation model is preferably the FPN model.

Since the image segmentation model needs to learn how to predict correct outputs from training data, a loss function is required to measure the difference between the predicted results of the image segmentation model and the ground truth results. Therefore, selecting an appropriate loss function has a significant impact on the performance and accuracy of the image segmentation mode.

In some implementations, the cross-entropy loss is selected as the loss function for the image segmentation model. Cross-entropy loss is a distribution-based loss function aimed at minimizing the difference between the model's predictions and the actual labels. As shown in Equation (8), ŷ represents the predicted value, and y represents the true value.

$${L}_{C E} ( y , \hat{y} ) = - ( y log ( \hat{y} ) + ( 1 - y ) log ( 1 - \hat{y} ) ) \qquad Equation ( 8 )$$

In some implementations, a Combo loss is selected as the loss function for the image segmentation model. The combo loss function is a combination of the Dice loss function and the Weighted Binary Cross-Entropy loss function. The combo loss primarily utilizes the flexibility of the Dice loss function to address class imbalance issues and incorporates the Weighted Binary Cross-Entropy loss function for curve smoothing. As shown in Equation (9), the overall loss function score is adjusted using the hyperparameter α and 1−α, where α ranges from 0 to 1. The hyperparameter β represents the weighted value for positive samples; when β>1, it reduces false negatives, and when β<1, it reduces false positives, helping to achieve better accuracy in scenarios with imbalanced training samples.

$${L}_{c o m b o} = α ( - \frac{1}{N} {\sum }_{i = 1}^{N} β ( y log ( {y}^{′} ) ) + ( 1 - β ) [ ( 1 - y ) log ( 1 - \hat{y} ) ] ) - ( 1 - α ) ( \frac{2 y \times \hat{y} + 1}{y + \hat{y} + 1} ) \qquad Equation ( 9 )$$

In some implementations, the accuracy of the image segmentation model may be evaluated using the Dice coefficient and Intersection Over Union (IoU). Both the Dice coefficient and IoU are metrics used to measure the similarity between the actual values and the predicted values. The value of the Dice coefficient is between 0 to 1, the value of the IoU is between 0 to 1, where higher value of Dice coefficient or IoU indicates greater similarity between the actual values and the predicted values.

In some implementations, Table 3 presents a comparison of results from using different image segmentation models for wound segmentation in an implementation of the present disclosure. As shown in Table 3, the accuracy of different image segmentation models (FCN, DeeplabV3+, FPN, or LinkNet) is compared. Among them, when the image segmentation model is FPN achieves the highest accuracy, with an IoU of 88.13% and a Dice coefficient of 92.72%.

TABLE 3

| Model | IoU | Dice coefficient |
|---|---|---|
| FCN | 83.12 ± 16.58 | 89.64 ± 12.70 |
| DeeplabV3+ | 85.70 ± 14.77 | 91.41 ± 11.92 |
| FPN | 88.13 ± 15.76 | 92.72 ± 11.69 |
| LinkNet | 86.62 ± 16.67 | 91.65 ± 13.39 |

### Tissue Segmentation Model

In some implementations, the processor may calibrate a plurality of images in the dataset that include the L-shaped color calibration card based on the L-shaped color calibration card to obtain a plurality of second preprocessed images.

In some implementations, the processor could perform image preprocessing on images in the dataset containing the L-shaped color calibration card to obtain second preprocessed images. Specifically, the method for performing second preprocessing on the images includes using the L-shaped color calibration card to conduct distortion correction and color calibration, converting the color space of the images, and applying denoising processing. The details of performing distortion correction, color calibration, color space conversion, and denoising processing using the L-shaped color calibration card are described in earlier sections and will not be repeated herein.

In some implementations, the processor may perform data augmentation on the plurality of second preprocessed images. The details of the data augmentation process are described in earlier sections and will not be repeated herein.

Moreover, in some implementations, the processor may further perform color quantization on the converted color space image. The reason is because when images remain in the RGB color space without color quantization, the large number of represented colors requires the tissue segmentation model to learn more color classifications, potentially leading to poor image classification results and prolonged training times. Based on the method presented by Godeiro et al. in the 2018 publication “Chronic wound tissue classification using convolutional networks and color space reduction”, the RGB colors in the images are converted into the CIELab color space, followed by reducing the complexity of multiple raw pixels in the images.

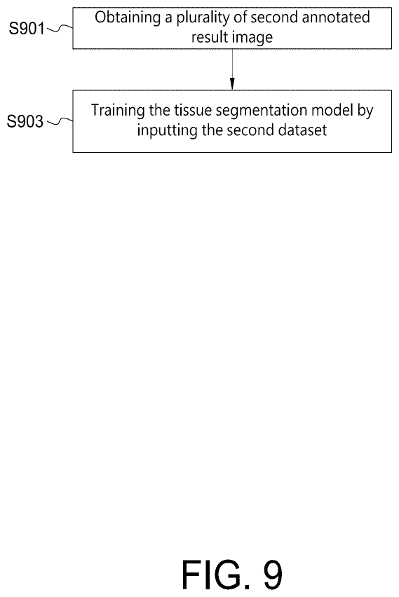

FIG. 9 is a flowchart of a method for establishing a tissue segmentation model according to an example implementation of the present disclosure.

Please refer to FIG. 9, in S901, obtaining a plurality of second annotated result images.

In some implementations, professional plastic surgeons may assist in annotating wound tissues in the plurality of second preprocessed images to generate a plurality of second annotated result images. The wound tissues in the second preprocessed images may represent wound tissues within the wound area. Specifically, the wound and non-wound areas in the images could be first segmented using superpixels. Then, the wound tissues within the wound area could be further segmented using superpixels. Labeling software such as Labelme or similar open-source software is employed to label different wound tissues within the wound area, assigning corresponding second label to obtain the plurality of second annotated result images. The second labels represent wound tissue type labels, including labels for granulation tissue, slough tissue, and eschar tissue. Accordingly, the second labels indicate the corresponding wound tissue types, such as granulation tissue, slough tissue, and/or eschar tissue.

FIG. 8B is a schematic diagram illustrating tissue segmentation according to an example implementation of the present disclosure.

Please refer to FIG. 8B. Superpixels are used to segment the wound area and non-wound area in the second preprocessed image 807, resulting in a distinguished wound area image 809, where the wound area within the second preprocessed image 807 is represented as region 8091. Subsequently, superpixels are further used to segment the wound tissues within region 8091. Wound tissue type labels (e.g., granulation tissue label, slough tissue label, and/or eschar tissue label) are then assigned to the wound tissues within region 8091, generating the second annotated result image 811. In some implementations, in the second annotated result image 811, region 8111 represents slough tissue in the second preprocessed image 807 and is labeled with the corresponding slough tissue label. Similarly, region 8113 represents eschar tissue in the second preprocessed image 807 and is labeled with the corresponding eschar tissue label.

In some implementations, the second annotated result images may include only the wound area and the wound tissues within the wound area, with the wound tissues assigned their corresponding second labels. The second labels could represent the wound tissue types corresponding to the respective wound tissues.

Please refer to FIG. 9, in step S903, training the tissue segmentation model by inputting the second dataset.

In some implementations, the processor may store the second annotated result images as a second dataset and further input the second dataset into a tissue segmentation model to train the tissue segmentation model using the second dataset. The purpose of the tissue segmentation model is to differentiate wound tissue types within the wound area in the images.

In some implementations, the tissue segmentation model may include Fully Convolutional Networks (FCN), the DeeplabV3+model, the LinkNet model, or the Feature Pyramid Networks (FPN) model. In some implementations, the tissue segmentation model is preferably the FPN model.

Since the image segmentation model needs to learn how to predict correct outputs from training data, a loss function is required to measure the difference between the predicted results of the tissue segmentation model and the ground truth results. Therefore, selecting an appropriate loss function has a significant impact on the performance and accuracy of the tissue segmentation model.

In some implementations, the cross-entropy loss is selected as the loss function for the tissue segmentation model. The details of the cross-entropy loss are described in the previous sections and will not be repeated herein.

In some implementations, a Combo loss is selected as the loss function for the tissue segmentation model. The details of the cross-entropy loss are described in the previous sections and will not be repeated herein.

In some implementations, the accuracy of the image segmentation model may be evaluated using the Dice coefficient and Intersection Over Union (IoU). Both the Dice coefficient and IoU are metrics used to measure the similarity between the actual values and the predicted values. The value of the Dice coefficient is between 0 to 1, the value of the IoU is between 0 to 1, where higher value of Dice coefficient or IoU indicates greater similarity between the actual values and the predicted values.

In some implementations, the accuracy of the tissue segmentation model in distinguishing the three types of wound tissues (granulation tissues, slough tissues, or eschar tissues) could be evaluated using the Overall IoU and Overall Dice coefficient. First, the average value for each type of wound tissue in a single image is calculated, the average value is then divided by the number of wound tissue types. When evaluating the accuracy of the tissue segmentation model across a plurality of images, the average IoU and average Dice coefficient for each image are first computed, and then their mean values are taken. This approach is used instead of directly summing up the average IoU and average Dice coefficient of the three tissue types, as shown in Equations (10) and (11). The advantage of this method providing a more objective evaluation of the accuracy of the tissue segmentation model, given that not all wound tissue images contain all three types of wound tissue.

$$Overall IoU = \frac{{\sum }_{i = 1}^{3} {IoU}_{i}}{class number} \qquad Equation ( 10 )$$

$$Overall dice coefficient = \frac{{\sum }_{i = 1}^{3} dice {coefficient}_{i}}{class number} \qquad Equation ( 11 )$$

In some implementations, Table 4 illustrates the results of training tissue segmentation models using different tissue segmentation models paired with different loss functions. As shown in Table 4, three types of tissue segmentation models (e.g., FCN, DeeplabV3+, FPN) were used in combination with two loss functions (Combo or CE) for tissue classification of wounds. Here, CE refers to Cross-Entropy loss, and Combo refers to Combo loss. By comparing the accuracy of these different tissue segmentation models, the most suitable combination of tissue segmentation model and loss function could be determined. From Table 4, it could be observed that the FCN and DeeplabV3+ models are more suited to using Cross-Entropy loss as the loss function. In contrast, the FPN model demonstrates better performance when paired with the Combo loss function. This is because the FPN model is designed to handle targets of different scales and sizes, and the Combo loss function effectively balances the targets of different scales and sizes, thus improving the accuracy of the tissue segmentation model.

TABLE 4

| | Loss | | Overall Dice |
|---|---|---|---|
| Models | functions | Overall IoU | coefficient |
| FCN | Combo | 61.75 ± 10.45 | 69.25 ± 10.60 |
| | CE | 64.56 ± 11.55 | 71.67 ± 11.45 |
| DeeplabV3+ | Combo | 64.49 ± 14.40 | 71.08 ± 14.04 |
| | CE | 69.81 ± 13.18 | 76.14 ± 11.90 |
| FPN | Combo | 74.80 ± 14.14 | 80.66 ± 12.13 |
| | CE | 69.10 ± 14.02 | 75.64 ± 12.40 |

In some implementations, Table 5 illustrates the results of training tissue segmentation models with or without the use of color calibration method. As shown in Table 5, the results demonstrate whether the use of a color calibration method improves the accuracy of tissue segmentation models. Compared to the FCN and DeeplabV3+ models, the FPN model achieved the highest accuracy. Specifically, the overall IoU and overall Dice coefficient of the FPN model were 74.80% and 80.66%, respectively, reflecting a 4.15% relative improvement in accuracy. These results indicate that training tissue segmentation models using color calibration images could enhance the accuracy of the tissue segmentation models.

TABLE 5

| | Color | | Overall | RI |
|---|---|---|---|---|
| | cali- | | Dice | (Relative |
| Models | bration | Overall IoU | coefficient | improvement) |
| FCN | No | 63.47 ± 10.63 | 70.93 ± 9.86 | — |
| | Yes | 64.56 ± 11.55 | 71.67 ± 11.45 | 1.68% |
| DeeplabV3+ | No | 68.99 ± 12.52 | 76.04 ± 11.01 | — |
| | Yes | 69.81 ± 13.18 | 76.14 ± 11.90 | 1.18% |
| FPN | No | 71.69 ± 12.68 | 77.94 ± 11.01 | — |
| | Yes | 74.80 ± 14.14 | 80.66 ± 12.13 | 4.15% |

In some implementations, Table 6 illustrates the results of training different tissue segmentation models with and without the application of a color quantization method. As shown in Table 6, when using three different tissue segmentation models (e.g., FCN, DeeplabV3+, FPN) with color quantification methods, the tissue segmentation models could all achieve high accuracy. Compared with the FCN and DeeplabV3+ models, the FPN model achieved the highest accuracy with an overall IoU of 74.80% and an overall Dice coefficient of 80.66%, reflecting a 3.08% relative improvement in accuracy. Furthermore, the FPN model could also reduce model training time by applying the color quantization method.

TABLE 6

| | Color | | Overall Dice | RI (Relative | Training |
|---|---|---|---|---|---|
| Models | quantification | Overall IoU | coefficient | improvement) | time (s) |
| FCN | No | 63.38 ± 13.67 | 70.63 ± 12.52 | — | 5120 |
| | Yes | 64.55 ± 11.55 | 71.67 ± 11.45 | 1.84% | 5132 |
| DeeplabV3+ | No | 69.76 ± 13.33 | 76.11 ± 12.10 | — | 3343 |
| | Yes | 69.81 ± 13.18 | 76.14 ± 11.90 | 0.07% | 3347 |
| FPN | No | 72.56 ± 14.29 | 78.61 ± 12.95 | — | 15098 |
| | Yes | 74.80 ± 14.14 | 80.66 ± 12.13 | 3.08% | 14100 |

In some implementations, images output by three different tissue segmentation models (e.g., FCN, DeeplabV3+, FPN) are used as a baseline, and post-processing (e.g., superpixels and conditional random fields) is performed on these images. The accuracy of the post-processed images is then compared. In some implementations, there are five post-processing methods, including (1) only using superpixels (referred to as “OB” in the present disclosure); (2) only using conditional random fields (referred to as “CRF” in the present disclosure); (3) applying conditional random fields first, followed by superpixels (referred to as “CO” in the present disclosure); (4) applying superpixels first, followed by conditional random fields (referred to as “OC” in the present disclosure); or (5) applying superpixels first, followed by conditional random fields, and then performing superpixel processing again (referred to as “OCO” in the present disclosure).

In some implementations, Table 7 illustrates the impact of post-processing methods on the accuracy of tissue segmentation models in an implementation of the present disclosure. As shown in Table 7, each of different tissue segmentation models (e.g., FCN, DeeplabV3+, FPN) combining with each of the five post-processing methods could effectively enhance the overall accuracy of the tissue segmentation models. In cases where the DeeplabV3+ or FPN model is used as compared to not using post-processing method, applying the CRF post-processing method improves the overall accuracy of the wound assessment method in an implementation of the present disclosure. Among these models, the FPN model demonstrates superior performance. When the FPN model is combined with the CRF post-processing method, its IoU improves from 74.80% to 76.72%, representing a 2.56% increase. Furthermore, utilizing the CRF method with the FPN model achieves higher accuracy, and the training time for CRF is relatively short. Therefore, adopting CRF as the post-processing method for the FPN model effectively enhances overall accuracy. Specifically, the FPN model demonstrates exceptional capabilities in feature extraction and feature fusion, enabling more accurate target segmentation during the post-processing step, which further enhances the overall performance of the FPN model.

TABLE 7

| | Post- | | Overall Dice | RI (Relative | Training |
|---|---|---|---|---|---|
| Model | processing | Overall IoU | coefficient | improvement) | time (s) |
| FCN | Base | 64.55 ± 11.55 | 71.67 ± 11.45 | — | — |
| | OB | 74.70 ± 13.42 | 80.74 ± 11.76 | 15.72% | 23.81 |
| | CRF | 74.30 ± 13.24 | 80.07 ± 11.45 | 15.10% | 0.76 |
| | CO | 75.65 ± 14.18 | 81.19 ± 12.38 | 17.19% | 25.02 |
| | OC | 74.69 ± 14.49 | 80.13 ± 13.11 | 15.70% | 23.83 |
| | OCO | 74.32 ± 14.12 | 79.95 ± 12.86 | 15.13% | 47.84 |
| DeeplabV3+ | Base | 69.81 ± 13.18 | 76.14 ± 11.90 | — | — |
| | OB | 75.13 ± 14.42 | 80.76 ± 12.34 | 7.62% | 24.16 |
| | CRF | 75.68 ± 13.94 | 80.87 ± 12.23 | 8.40% | 0.75 |
| | OC | 75.15 ± 14.93 | 80.43 ± 13.13 | 7.64% | 24.51 |
| | CO | 74.96 ± 15.17 | 80.05 ± 13.24 | 7.37% | 20.68 |
| | OCO | 74.33 ± 15.39 | 79.48 ± 13.73 | 6.47% | 41.52 |
| FPN | Base | 74.80 ± 14.14 | 80.66 ± 12.13 | — | — |
| | OB | 76.14 ± 14.26 | 82.05 ± 11.91 | 1.79% | 21.20 |
| | CRF | 76.72 ± 15.07 | 82.13 ± 12.76 | 2.56% | 0.66 |
| | CO | 75.80 ± 14.17 | 81.60 ± 11.93 | 1.33% | 71.77 |
| | OC | 75.41 ± 14.64 | 80.90 ± 12.69 | 0.81% | 21.57 |
| | OCO | 75.99 ± 14.92 | 81.69 ± 12.72 | 1.58% | 43.37 |

In summary, to validate the accuracy of the tissue segmentation model in an implementation of the present disclosure, a comparison was conducted between models trained directly with original images and models trained with color-calibration images. Using the same dataset, model, and parameters, the results are shown in Table 8. As shown in Table 8, when the model is trained directly with original images, the accuracy of tissue classification (overall IoU and overall Dice coefficient are 62.21% and 70.81%, respectively) is significantly lower than the accuracy of the tissue segmentation achieved by the tissue segmentation model that is proposed in one implementation of the present disclosure (overall IoU and overall Dice coefficient are 76.72% and 82.13%, respectively). The tissue segmentation model proposed in one implementation of the present disclosure effectively improves the accuracy of tissue segmentation by 23.32% compared to models trained directly using original images (compared approach). The reason lies in the training data, used by the tissue segmentation model that is proposed in one implementation of the present disclosure, consists of images with non-wound areas removed. This processed training data is then used to train the tissue segmentation model. The proposed approach enables the tissue segmentation model to focus exclusively on the tissues within the wound area, allowing the tissue segmentation model to more effectively learn the features of wound tissues within the wound area. Such proposed approach helps the tissue segmentation model concentrate on learning the features of wound tissues inside the wound area, thus reducing interference from non-wound regions in the images during the model's learning process.

Furthermore, the training data utilized in one implementation of the present disclosure consists of images that have been distortion corrected and color calibrated using the L-shaped color calibration card, followed by denoising processing of these distortion corrected and color calibrated images. Finally, the images output from the tissue segmentation model undergo post-processing to enhance the overall accuracy of the tissue segmentation model. Therefore, it could be concluded that the method proposed in one implementation of the present disclosure enables the tissue segmentation model to more precisely segment the wound tissues within the wound area and to determine the wound tissue categories and the distribution of wound tissues from the wound tissue images.

TABLE 8

| | | | Overall Dice | RI (Relative |
|---|---|---|---|---|
| Model | Approach | Overall IoU | coefficient | improvement) |
| FPN | Compared | 62.21 ± 11.86 | 70.81 ± 11.83 | — |
| | approach | | | |
| | Proposed | 76.72 ± 15.07 | 82.13 ± 12.76 | 23.32% |
| | approach | | | |

In some implementations, Table 9 presents the accuracy of different tissue segmentation models. In Table 9, the Dice coefficient method is used to evaluate overall accuracy. Specifically, “Pholberdee” refers to the method presented in “Feature Pyramid Networks for Object Detection,” published by N. Pholberdee et al. in 2018; “Niri” refers to the method presented in “A Superpixel-Wise Fully Convolutional Neural Network Approach for Diabetic Foot Ulcer Tissue Classification,” published by R. Niri et al. in 2021; and “Ramachandram” refers to the method presented in “Fully Automated Wound Tissue Segmentation Using Deep Learning on Mobile Devices: Cohort Study,” published by D. Ramachandram et al. in 2022. The method of study of present disclosure represents the image evaluation method proposed in one implementation of the present disclosure. The results indicate that, compared to the methods proposed by Pholberdee, Niri, and Ramachandram, the image evaluation method proposed in one implementation of the present disclosure achieves an overall accuracy of 82.13%, significantly higher than the other three methods.

TABLE 9

| | | | | Overall |
|---|---|---|---|---|
| | | Number | Image | accuracy |
| Paper | Approach | of images | source | (%) |
| Pholberdee | CNN | 180 | Medetec | 57.02 |
| Niri | FCN | 219 | ESCALE | 75.74 |
| Ramachandram | Auto tissue | 58 | Swift | 61.00 |
| The study of | FPN | 453 | NCKUH | 82.13 |
| present disclosure | | | | |

As described above, the wound assessment system and the wound assessment method of the present disclosure could accurately analyze the target wound image data of a subject, and effectively evaluate the wound area and wound tissue type of the subject based on the target wound image. This, in turn, aids in early planning for subsequent medical treatments, thus reducing the risk of complications caused by wounds not being promptly treated or improperly treated.

The embodiments shown and described above and below are only examples. Many details are often found in the art. Therefore, many such details are neither shown nor described herein for the sake of brevity. Even though numerous characteristics and advantages of the present disclosure have been set forth in the foregoing description, together with details of the structure and function of the present disclosure, the present disclosure is illustrative only, and changes may be made in the details. It will therefore be appreciated that the embodiments described above and below may be modified within the scope of the claims.

What is claimed is:

1. A wound assessment method, comprising:
receiving an image, the image comprising an L-shaped color calibration card and a wound;
performing an image preprocessing on the image, based on the L-shaped color calibration card, to obtain an adjusted image, the image preprocessing comprising a distortion correction and a color calibration;
obtaining a wound image excluding the L-shaped color calibration card, based on the adjusted image, the wound image comprising a plurality of regions; and
classifying each of the plurality of regions of the wound image into one of wound tissue types to obtain a wound tissue type distribution of the wound, the wound tissue types comprising a granulation tissue, a slough tissue, and an eschar tissue.

2. The wound assessment method of claim 1, wherein the wound is located on an open side of a L-shaped structure ruler of the L-shaped color calibration card in the image.

3. The wound assessment method of claim 1, wherein
the distortion correction is performed on the image based on an angle of the L-shaped color calibration card in the image.

4. The wound assessment method of claim 1, wherein the L-shaped color calibration card comprises an L-shaped structure ruler and a plurality of color calibration elements, the plurality of color calibration elements comprises a red pattern, a yellow pattern, a blue pattern, a green pattern, and four progressive grayscale patterns.

5. The wound assessment method of claim 4, wherein
the color calibration is performed on the image based on the red pattern, yellow pattern, blue pattern, and green pattern of the L-shaped color calibration card in the image.

6. The wound assessment method of claim 4, further comprising:
performing a white balance on the image using the four progressive grayscale patterns.

7. The wound assessment method of claim 1, further comprising:
converting a color space of the adjusted image to obtain a converted color-space adjusted image;
performing a color quantization on the converted color-space adjusted image to reduce a complexity of raw pixels in the converted color-space adjusted image and obtain a color-quantized adjusted image; and
performing a denoising processing on the color-quantized adjusted image.

8. The wound assessment method of claim 1, further comprising:
inputting the adjusted image into an image segmentation model to obtain the wound image excluding the L-shaped color calibration card;
inputting the wound image into a tissue segmentation model to obtain a wound tissue image; and
determining the wound tissue type distribution of the wound based on the wound tissue image and the wound tissue types.

9. The wound assessment method of claim 8, further comprising:
performing an area evaluation on the wound tissue image based on the L-shaped color calibration card.

10. The wound assessment method of claim 8, further comprising:
optimizing the wound tissue image by applying a conditional random field.

11. The wound assessment method of claim 8, wherein a method for establishing the image segmentation model comprises:
obtaining a plurality of first annotated result images, the plurality of first annotated result images comprising a plurality of first preprocessed images, each of the plurality of first preprocessed images comprising a wound category label and a non-wound category label;
storing the plurality of first annotated result images as a first dataset; and
training the image segmentation model by inputting the first dataset, wherein the plurality of first preprocessed images is corrected based on the L-shaped color calibration card.

12. The wound assessment method of claim 8, wherein the image segmentation model comprises a Feature Pyramid Network (FPN).

13. The wound assessment method of claim 8, wherein a method for establishing the tissue segmentation model comprises:
obtaining a plurality of second annotated result images, the plurality of second annotated result images comprising a plurality of second preprocessed images, each of the plurality of second preprocessed images comprising at least one of wound tissue type labels, wherein the wound tissue type labels comprise a granulation tissue label, a slough tissue label, and an eschar tissue label;
storing the second annotated result images as a second dataset; and
training the tissue segmentation model by inputting the second dataset, wherein the plurality of second preprocessed images is corrected based on the L-shaped color calibration card.

14. The wound assessment method of claim 8, wherein the tissue segmentation model comprises a Feature Pyramid Network (FPN).

15. The wound assessment method of claim 1, further comprising: calculating a wound healing score based on the wound image.

16. A wound assessment system, comprising:
at least one processor; and
at least one memory coupled to the at least one processor and storing at least one computer-executable instruction that, when executed by the at least one processor, cause the wound assessment system to execute the wound assessment method of claim 1.
